(19) United States
(12) Patent Application Publication
(10) Pub. No.: US 2008/0196533 A1
(43) Pub. Date: Aug. 21, 2008

(54) Remotely Actuated Robotic Wrist

(76) Inventors: Massimo Bergamasco, Castelmaggiore-Calci (IT); Fabio Salsedo, Latina (IT); Stefano Spinelli, Lucca (IT); Marco Fontana, Pisa (IT)

Correspondence Address: DENNISON, SCHULTZ & MACDONALD, 1727 KING STREET, SUITE 105, ALEXANDRIA, VA, 22314, US

(21) Appl. No.: 10/579,475

(22) PCT Filed: Nov. 15, 2004
(86) PCT No.: PCT/IB04/03731

§ 371 Date: Jun. 6, 2006

(30) Foreign Application Priority Data
Nov. 14, 2003 (IT) P2003A000107

(51) Int. Cl. B25J 17/02; A61B 19/00

(52) U.S. Cl. 74/490.06; 901/23; 901/29

(57) ABSTRACT

Remotely actuated robotic wrist for applications in the field of teleoperation, for example for mininvasive surgical operations, comprising a distal element (3) mounted on a support (2) capable instantaneously to rotate with respect to a fixed member (5), for example by a ball joint (10) that allows three rotational degrees of freedom. In particular, the support (2) can be oriented with respect to the fixed member (5) with a redundant actuating system by arranging four forces in eccentric points, for example by means of tendons (8), and causing the rotation of the support (2) about the central post (5) by the ball joint (10). Alternatively, the support (2) can be oriented with respect to the fixed member (5) by a mechanism that reproduces the rolling of a mobile sphere, which belongs to the support, on a fixed sphere, integral to the fixed member.

### FIELD OF THE INVENTION

The present invention relates to robotics and teleoperation and in particular it relates to a remotely actuated robotic wrist capable of transmitting a feedback force on an operator.

For example, the wrist can be used in Computer Aided Surgery, and particularly in mininvasive surgery, where the wrist can be mounted on a manipulator arm of a surgical robot remotely actuated by an operator (teleoperation surgery) or it can be used as distal component of a laparoscopic active instrument.

### DESCRIPTION OF THE PRIOR ART

In the field of robotics and advanced teleoperation the problem is felt of a remotely actuated robotic wrist producing a feedback force on the operator. The desired features of a wrist for such an application are its easy construction, a relatively low cost and maximum operative flexibility of the wrist and of a possible distal member, in order to cover the maximum allowable degrees of freedom.

In one of the possible applications, the mininvasive surgery, it is necessary to carry out a surgical operation, for example in the abdomen or in the thorax of a patient, using small and thin instruments and an endoscope introduced in the human body, minimizing the size of the cut necessary to access the surgical site. The images detected by the endoscope are shown on a monitor where the surgeon can watch the surgical site in real time and execute the required operations.

One among the mininvasive techniques most common is the laparoscopy, whose success is due to the many advantages that it offers with respect to traditional surgery, such as less traumatic consequences on the patient, shorter hospitalization, and reduction of the risk of infections. Normally, mininvasive techniques also have the advantage of reducing the sanitary costs.

Mininvasive surgery can be effected successfully, either in a manual way, or with the aid of a robotic apparatus, also called slave, having manipulator arms remotely actuated by the surgeon through a special interface, also called master. This way, a surgeon acting on the master can carry out a surgical operation even at considerable distance from the patient where the slave holding the surgical instruments is arranged.

In the last few years different researches have developed the surgical instruments up to achieving high performances concerning reliability, precision and the safety of mininvasive operations.

In particular, surgical heads have been developed, to be mounted at the end of either an endoscope or a laparoscopic “trocar” for handling the tissues to treat in the abdomen of the patient.

Two main types exist of surgical heads for mininvasive operations.

A first type follows the principle of arranging the actuators (electric, hydraulic, pneumatic) and the possible sensorization of the head same. In this way the head is independent, so to say, from the external world, except from tendons that provide the control and feedback signals. This solution, however, is structurally complex concerning the assembling steps, is heavy and has high costs owing to the miniaturization of its components. In fact, the typical size of a head of this type is between 10 and 12 mm.

A second kind of surgical heads arranges the motors and sensors outside the head. This solution has different advantages among which a much easier assembling step owing to the lower number of components, low inertia, free choice of the actuators for the absence of housing constraints, as well as an easy sterilization, since the motors and the sensors are external. However, also the surgical robotic heads belonging to the latter kind have to be, in any case, systematically sterilized by specialized operators, and involve then high costs since the hospitals must obtain instruments in a larger amount in order not to await that the instruments to be sterilized are ready.

A milli-robotic head belonging at the second kind has been made by the Berkeley University. It has a structure very easy comprising two metal platforms united by a central spring that works as spherical hinge. The head woks with three tendons operated by corresponding motors, located out of the head same. The distal instrument extends from a central channel of the upper platform, whereas the CCD lenses, the optical fibres, and possible tubes for irrigating the tissues or for cauterization are arranged laterally. A type of robotic head of this kind has 2 degrees of freedom, and in particular two rotations with respect to axes normal to the axis of the instrument and the operation is redundant.

Various solutions have been presented for implementing also the rotation about the central axis, considered relevant by the surgeons since it allows to execute some essential manoeuvres, which otherwise would require torsions/rotations of the whole endoscope. A possible solution provides a central pulley operated by an additional tendon that causes the rotation of the upper platform. This result is achieved through a plurality of pulleys that orient the tendon. This solution, even if easy and functional, has limits due to the friction between the bushings where the tendons slide, and by the numerous pulleys necessary, which introduce relevant assembling problems given the small size of the head, about 10 mm.

A second solution provides a chain of platforms connected to each other through pivot joints. The operation of this mechanism is carried out through some tendons that pass through the holes of said platforms. Even in this case the solution is easy and reduces remarkably the costs, but friction occurs where the tendons slide on the surfaces of the holes. Normally, furthermore, the instruments presently existing do not allow the transmission of a feedback force on the surgeon, i.e. they are not capable to reflect “haptic” sensations relative to the contact. This affects the diffusion of robotic surgery, owing to the impossibility of transmitting to the surgeon such sensations, precluding control of the forces applied by the end effector on the tissues, thus increasing remarkably the risk of errors.

### SUMMARY OF THE INVENTION

It is a feature of the present invention to provide a remotely actuated robotic wrist for robotic and teleoperation applications that provides a maximum flexibility.

It is a particular feature of the invention to provide such a remotely actuated robotic wrist suitable for supporting and manoeuvring an instrument for mininvasive surgical operations that is structurally easy and cost effective.

It is another feature of the present invention to provide a remotely actuated robotic wrist that allows three degrees of freedom of orientation of the instrument, or two degrees of freedom with the maximum coverage of the field of action of the same.

It is also a feature of the present invention to provide a robotic wrist that allows, in addition to the orientation, to manoeuvre the opening-closing action of the end effector, such as a gripper, a cutter, etc.

It is, furthermore, a feature of the present invention to provide a robotic wrist with a sufficiently precise feedback of the forces applied by the end effector through a return force on the operator, raising the rate of precision of the operation.

It is a further feature of the present invention to provide a robotic wrist suitable for a production of plastic material for a disposable application.

These and other features are accomplished with one exemplary remotely actuated robotic wrist according to the invention, whose characteristic is that it comprises:
at least a distal element;
an orientable support integral to said distal element;
a fixed member having a pivot about which said support is capable instantaneously to rotate;
remote means with respect to said distal element for creating at least two independent forces suitable for causing said support to move with respect to said pivot according to at least two independent directions;
deviating means said at least two forces so that they are applied to said support according to two predetermined positions.

Further characteristics of the invention are defined by the attached claims, according to independent claim 1.

### BRIEF DESCRIPTION OF THE DRAWINGS

The invention will now shown with the following description of an exemplary embodiment thereof, exemplifying but not limitative, with reference to the attached drawings wherein:

FIG. 1 shows a perspective view of a robotic wrist for mininvasive surgical operations, according to the invention;

FIG. 2 shows a perspective view of a possible exemplary embodiment of connecting arm for deviating the means for actuating the support of the robotic wrist of FIG. 1;

FIG. 3 shows a perspective view of a possible exemplary embodiment of a base used as support for the connecting arms of FIG. 2;

FIGS. 4 and 5 show an elevational front view of a ball joint respectively in exploded and assembled configuration;

FIGS. 6 and 7 show diagrammatically the actuating mechanism of the robotic wrist of FIG. 1;

FIG. 8 shows a perspective view of a device for mininvasive surgical operations, according to the invention;

figures from 9 to the 12 show diagrammatically a perspective view of four possible positions of the robotic wrist of FIG. 1;

figures from 13 to 16 show a perspective top plan view side view of a possible exemplary embodiment for generating the force and transmission of the movement used for operating the device of FIG. 8;

FIGS. 17 and 18 show a diagrammatical view for operating the instrument mounted on the robotic wrist of FIG. 1;

FIGS. 19 and 20 show a top plan view of an instrument to be mounted on the robotic wrist of FIG. 1.

In figures from 21 to 25 a diagrammatical view is shown of the kinematic operation of an alternative exemplary embodiment of the remotely actuated robotic wrist according to the invention;

FIG. 26 shows an alternative embodiment of the diagrammatical kinematical view of FIGS. 21-25, with decomposition of the movement of two spheres rolling on each other by means of two kinematical chains;

FIG. 27 shows a simplified embodiment of the diagrammatical view of the kinematics of FIG. 26;

FIGS. 28 and 29 show a practical embodiment of a robotic wrist like that of FIG. 27 in two operative positions.

### DESCRIPTION OF THE PREFERRED EXEMPLARY EMBODIMENT

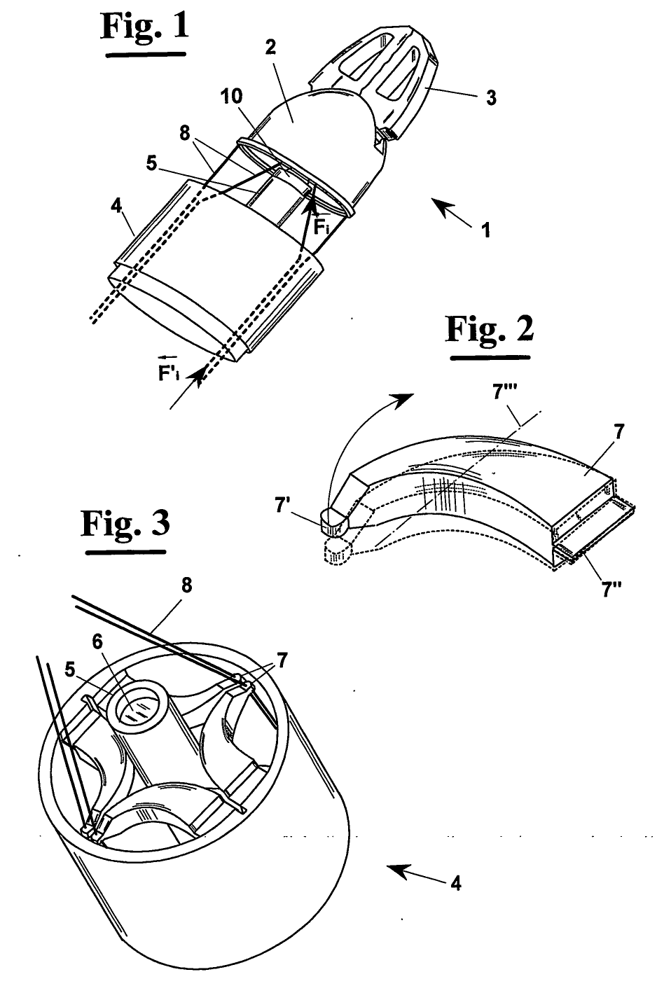

In FIG. 1 a robotic wrist 1 is shown for mininvasive surgical operations carried out through not shown “slave” manipulators remotely actuated by an operator, according to the present invention.

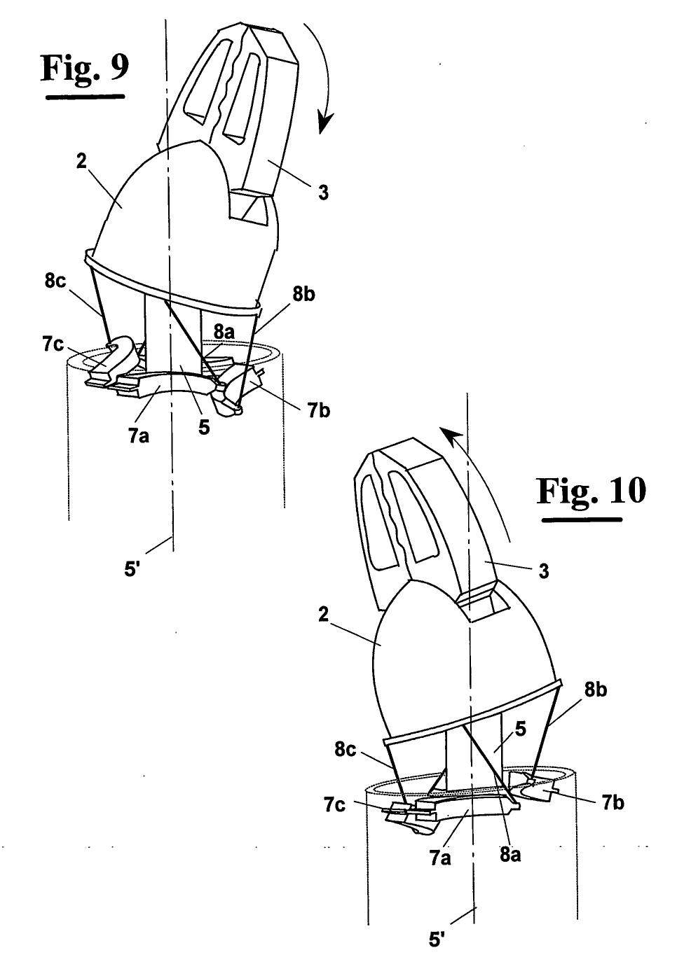

A robotic wrist 1 comprises a distal member as an end effector 3 mounted on a support 2 pivotally connected to a central post 5 integral to a fixed base 4, for example by a ball joint 10 that allows three rotational degrees of freedom (FIG. 4). This has a circular portion 12 housed with possibility of rotating in a housing 11 and an elongated portion 13 that in operative conditions is oriented towards the end effector 3.

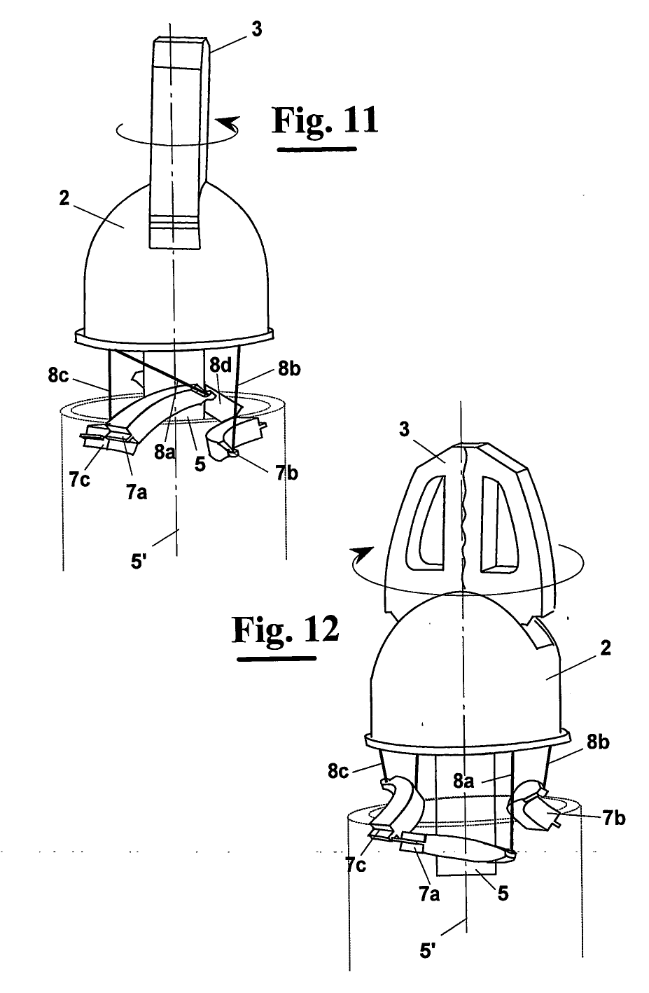

In particular, support 2 can be oriented with respect to central post 5 with a redundant actuating system, by arranging four forces F₁-F₄ in eccentric points P₁-P₄, for example by means of tendons 8, and causing support 2 to rotate about central post 5 by ball joint 10 (FIG. 6).

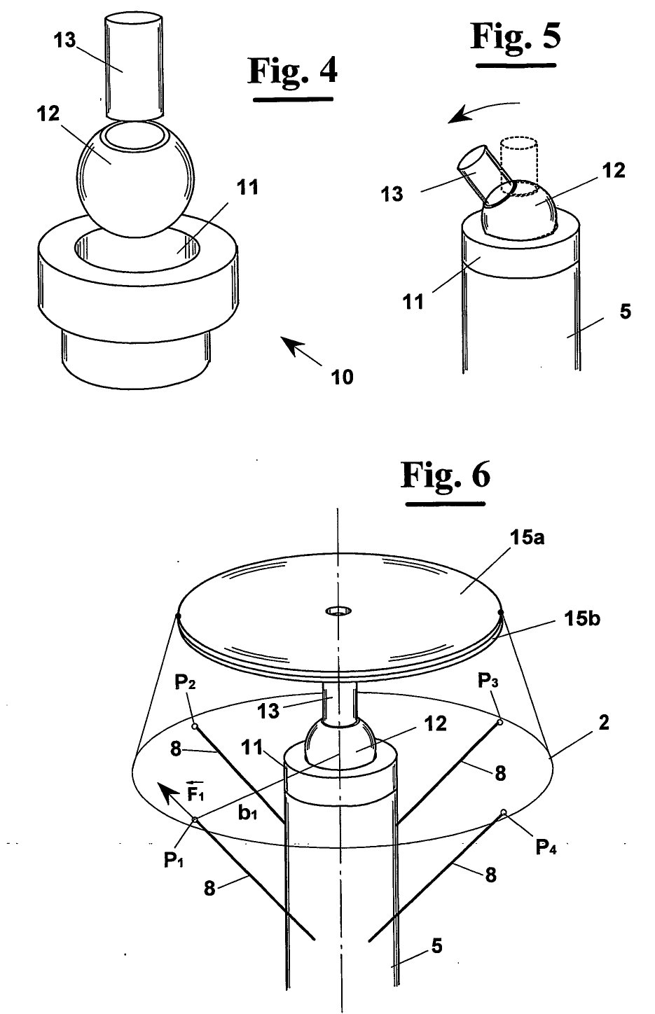

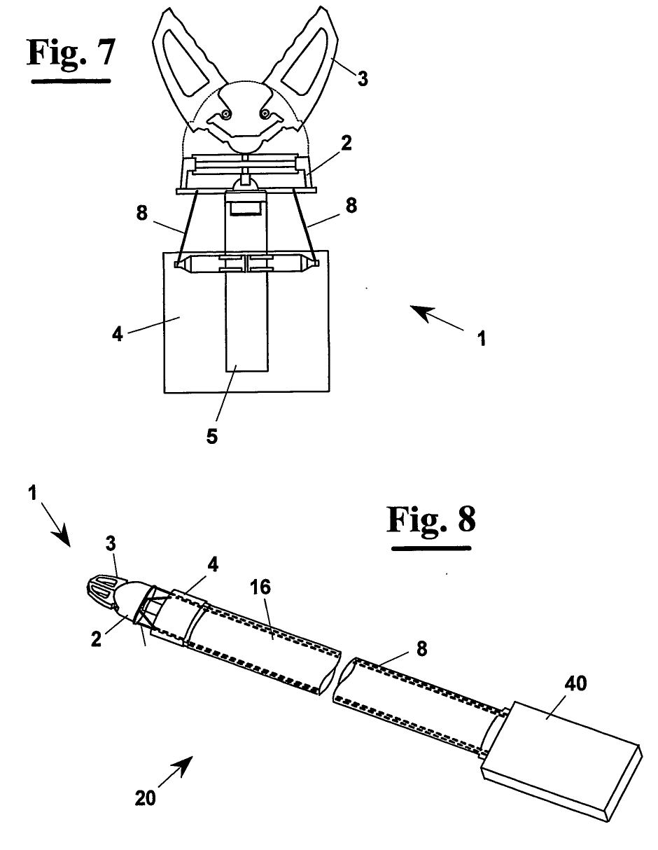

The direction of application of forces F₁-F₄ is determined by connecting arms 7 (FIGS. 2 and 3), which deflect forces F₁-F₄ generated by a motor 40 located upstream and described hereafter (FIG. 1). In an exemplary embodiment shown in FIG. 2 connecting arms 7 are cantilevers that have a central body, of relatively high thickness, shaped as a tapering arc with an end 7′ and a fixed joint 7″, with a cross section relatively thin that extends from the body of fixed base 4. This geometry allows a high flexibility in a preferential plane and high stiffness in other planes. This way, it is possible to provide a transmission of the movement with low friction and, therefore, to increase the precision of determination of the force applied by the instrument in the surgical site.

In the exemplary embodiment of FIG. 2 four connecting arms 7 are provided having a fixed joint 7″ connected to the body of the base 4 and a free end 7′ that under a force F′ rotates with respect to a resilient axis 7′″ of the fixed joint cross section. This way, a compact structure is achieved and with minimum encumbrance, made of plastic material, for example TPE, particularly indicated for being used as disposable device.

In case the instrument mounted on the robotic wrist 1 has an opening/closing mechanism, such as a surgical gripper 3, between the instrument and the elongated portion 13 of ball joint 10 means with controlled yield 15a and 15b are provided (FIG. 6).

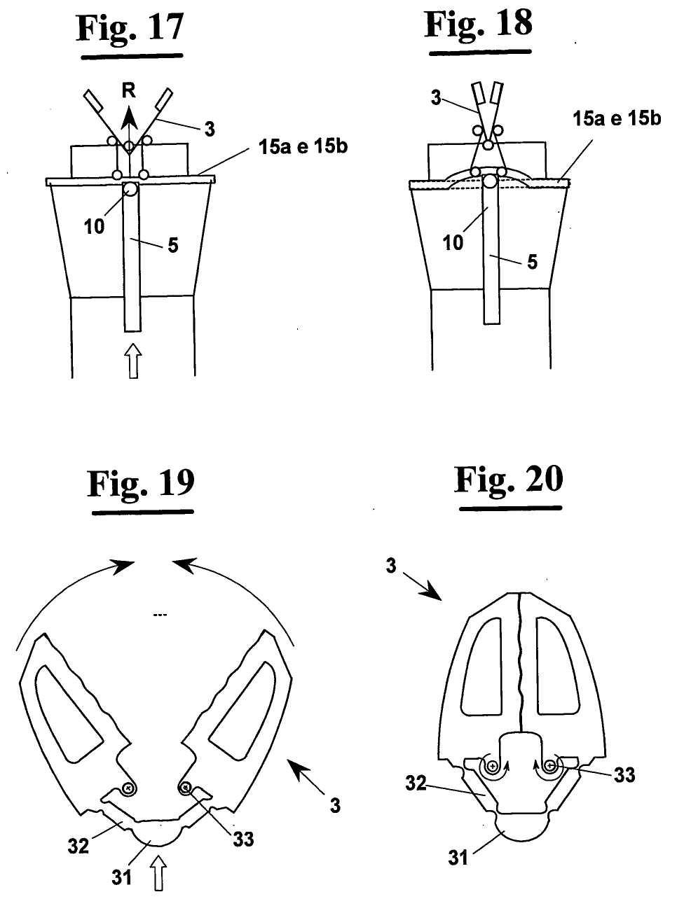

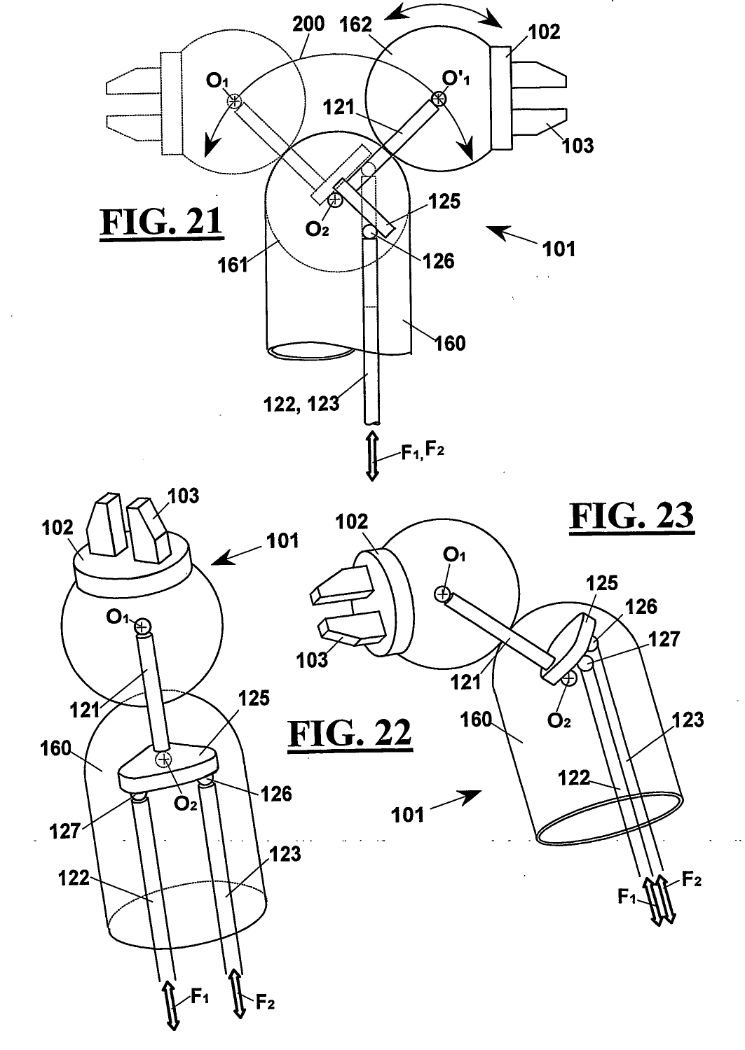

More in detail, when tendons 8 are subject to a tension higher than a determined value, the resultant of the reaction force of the ball joint 10 on support 2, and in particular its component R in the orthogonal direction to the plane of points P1-P4, causes a controlled deformation (bending) of the means 15a and 15b (FIGS. 17-20). Therefore, beyond a certain value of component R, the amount of the deformation of the means 15a and 15b is such that the elongated part 13 of the ball joint 10 contacts base 31 of gripper 3. Beyond this value the two parts that form the gripper 3 begin to rotate about each fulcrum 33, closing the gripper. Any further increase of the load on basis 33 allows to adjust both of the position and the force acting on the tissues allowing an accurate control thereof. Owing to the redundancy of the actuating system of support 2 it is possible to activate the end effector without changing the orientation of support 2.

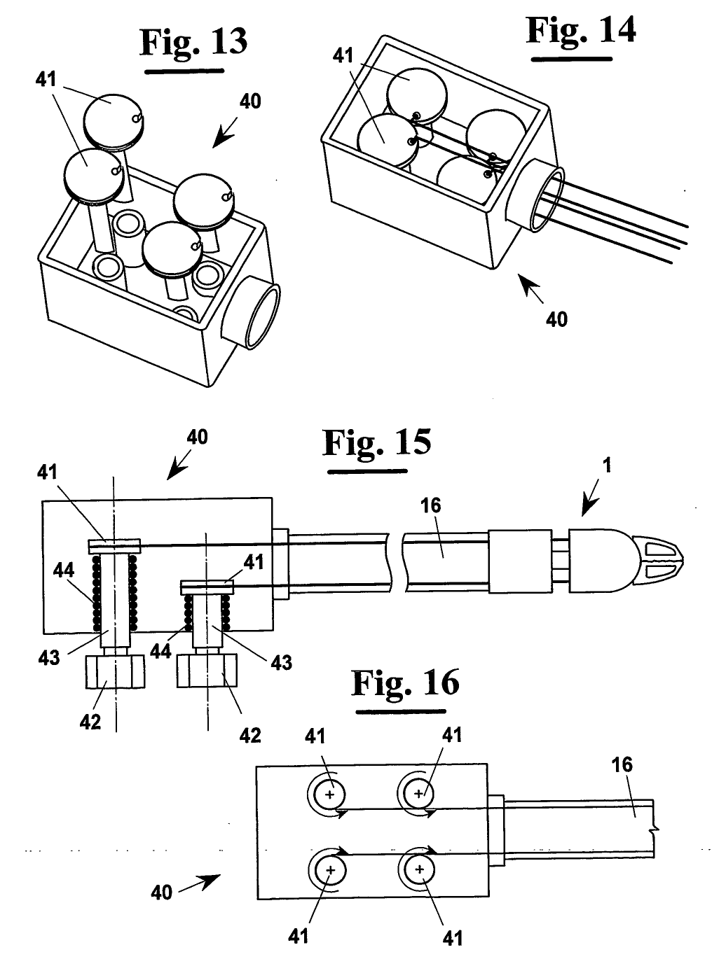

The robotic wrist 1, as above described, can be mounted on a trocar 16 of known art, where tendons 8 extend and transmit the force F′, generated by a motor 40 and suitably deflected by connecting arms 7, to the robotic wrist of a device 20, which can carry out mininvasive surgical operations (FIG. 8).

In figures from 9 to 12 four possible orientations are shown of robotic wrist 1 obtained acting onto tendons 8a-8d and then onto the respective connecting arms 7a-7d, following predetermined kinematic schemes.

In particular, tendons 8a-8d are subject to a tension, and changing each respective tension it is possible to cause the rotation of robotic wrist 1 in one of the three planes corresponding to the degrees of freedom of ball joint 10.

In figures from 13 to 16 the interface of connection 40 is shown of Tendons 8 to the respective motors 42. It provides a pulley 41 having a stem 43 directly fitted on the shaft of the respective motor 42. In particular, each pulley 41 is mounted on a bearing and is associated to a spring 44 to it co-axial suitable for pre-tensioning tendons 8. Sensors of position, for example encoders, can be mounted integral to the shafts of motors 42, with which it is possible to determine the position of the robotic wrist 10 and of connecting arms 7. In another preferred embodiment it is possible to provide a releasable connection between the shafts of the motors 42 and the stems 43 of the pulleys by means of clutches, for example. This way a device 20 is obtained for mininvasive surgical operations completely passive reducing the costs and reducing the sterilization problems.

In figures from 21 to 25 a diagrammatical kinematical view is shown of an alternative exemplary embodiment of the remotely actuated robotic wrist shown in figures from 1 to 20. As shown in FIG. 21, the mechanism of the wrist 101 is equivalent to two spheres, or portions of sphere, rolling on each other. More in detail, the fixed pivot O₂ is located at the centre of first sphere 161, belonging to fixed member 160, and is connected by an arm 121 to the centre O₁ of second sphere 162. This way, the centre O₁ describes a circular trajectory 200 with respect to fixed pivot O₂ having radius equal to the length of arm 121. The motion of second sphere 162 with respect to first sphere 161 is caused by remote motor means, not shown, whose movement and the relative forces are transmitted by a kinematik system comprising a platform 125 movable pivotally about fixed pivot O₂. In particular, platform 125 is operated by the motor means through a first stick 123 that ends at a hinge 126 of platform 125 and a second stick 122 that ends at a hinge 127 of platform 125 (FIGS. 22-24). According to the intensity and the direction of the force applied to the sticks 122 and 123, the platform 125 moves instantly in a plane oriented with respect to sphere 161. A following rotation of support 102 with respect to pivot O₂ allows to arrange the distal member 103 in a desired operative position. In other words, the overall movement of the distal member 103 can be seen as the combination of a first rotation about fixed pivot O₂ and a second rotation about point O₁.

In FIG. 25 the possibility is shown causing distal member to follow an angular trajectory of 360°, from position 103 to position 103″, by choosing a suitable ratio between the radius of spheres 161 and 162, for example 1 to 2, and therefore, the gear ratio of the movement.

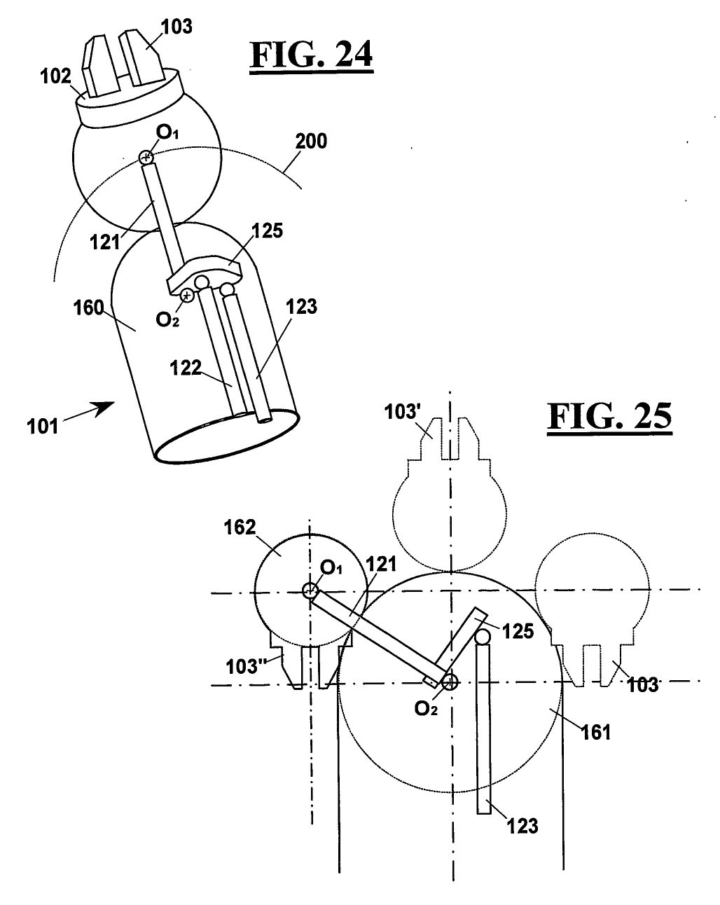

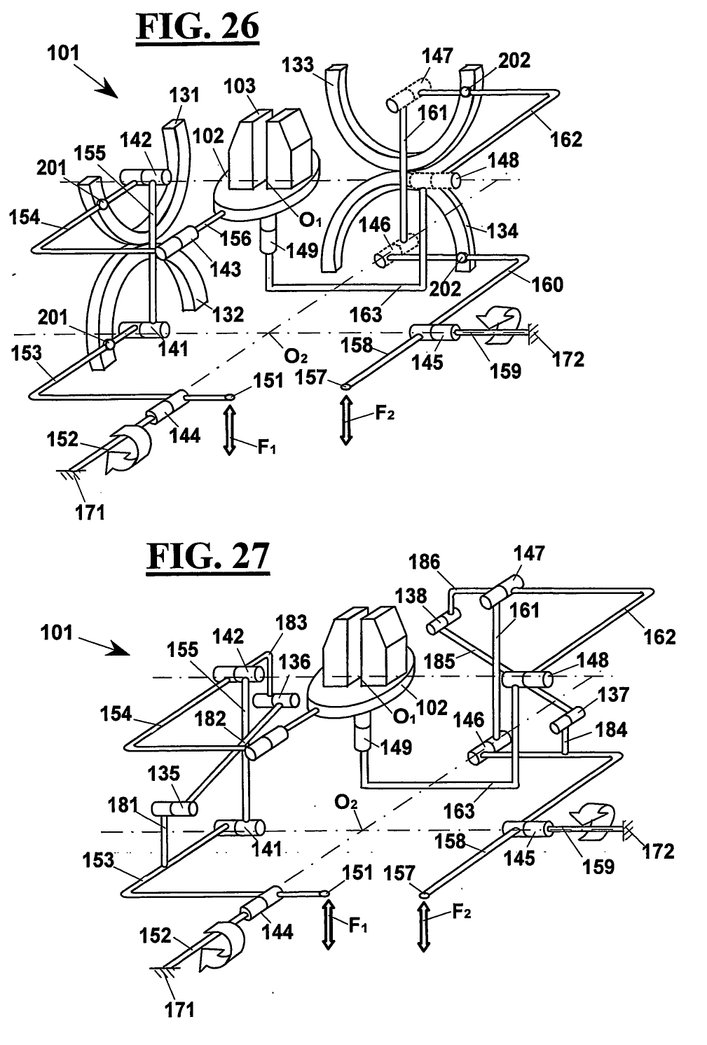

What above described represents the operation of an exemplary embodiment of the remotely actuated robotic wrist 101, whose practical implementation is shown as an alternative exemplary embodiment in FIGS. 26 and 27.

In particular, the rolling movement of sphere 161 on sphere 162 is split in two contributions in two respective orthogonal planes, using the mechanism described hereafter. More in detail, in the exemplary embodiment of FIG. 26, the transmission of the movement is obtained from a first kinematical chain comprising a plurality of stiff elements 152-156 connected by means of pivot joints 141-143 and a couple of gears 131 and 132 that works in combination with a second kinematical chain, comprising a plurality of stiff elements 158-163 connected by means of pivot joints 146-149 and a couple of gears 133 and 134. Wheels 131 and 132 are connected to the first kinematical chain in respective points 201 and 202 and have centre integral to respective hinges 141 and 142. Similarly, wheels 133 and 134 are connected to the second kinematical chain in respective points 203 and 204 and have a centre integral to the respective hinges 146 and 147.

The independent forces F1 and F2 that are transmitted through each kinematical chain to support 102 are generated by respective remote motors, not shown, and are applied to the relative kinematical chain at points 151 and 157 respectively. This produces the motion of the kinematical chain with respect to fixed points 171 and 172 of device 101, which points belong, along with fixed pivot O₂, to the fixed member of the device. In an exemplary embodiment of FIG. 26 the distance between the points O₁ and O₂ represents an invariant of the system since it coincides with the length of the stiff elements 155 and 161 of the two kinematical chains, which is also the distance between the centres of the two couples of gears 131, 132 and 134, 135.

In FIG. 27 an exemplary embodiment is shown of the robotic wrist 101 alternative to that of FIG. 26. The operation of the two exemplary embodiments is the same, but in the embodiment of FIG. 27, instead of the couples of gears 131-132 and 133-134 of the embodiment of FIG. 26, a tern of stiff elements 181-183 and 184-186 is provided instead, which are interconnected by pivot joints 135-136 and 137-138 respectively.

The two exemplary embodiments of FIGS. 26 and 27 are particularly advantageous because replace practically the mechanism of FIGS. 21-25 and do not cause interferences between the many stiff elements, or links, which make them up.

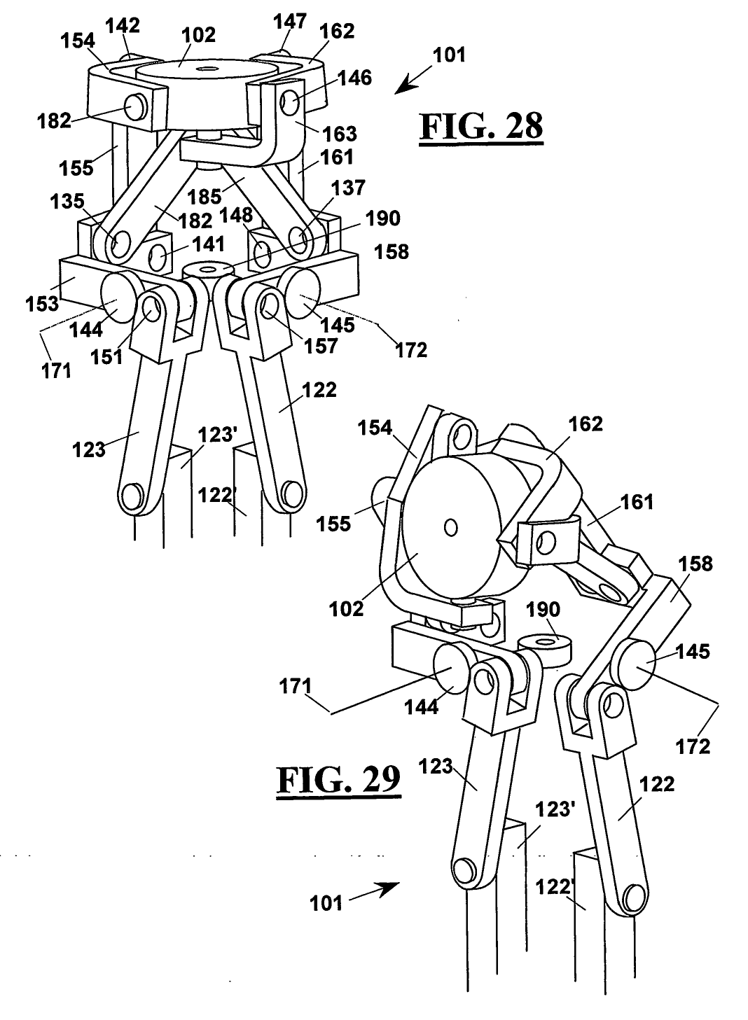

Another practical embodiment of the mechanism of FIG. 27 is shown by the robotic wrist 21 of FIGS. 28 and 29. The parts of FIGS. 28 and 29 have the same numbers of the parts of FIG. 27 since have the same functions. In FIGS. 28 and 29 is shown a sliding hole 190 allows the motion of one or more tendons for operating a distal member 103. This is allowed thanks to the absence of interference between the links which actuate the support 102 and the central zone of the device.

The foregoing description of a specific embodiment will so fully reveal the invention according to the conceptual point of view, so that others, by applying current knowledge, will be able to modify and/or adapt for various applications such an embodiment without further research and without parting from the invention, and it is therefore to be understood that such adaptations and modifications will have to be considered as equivalent to the specific embodiment. The means and the materials to realise the different functions described herein could have a different nature without, for this reason, departing from the field of the invention. It is to be understood that the phraseology or terminology employed herein is for the purpose of description and not of limitation.

1. Remotely actuated robotic wrist, characterized in that it comprises:
at least a distal element;
an orientable support integral to said distal element;
a fixed member having a pivot about which said support is capable instantaneously to rotate;
remote means with respect to said distal element for creating at least two independent forces suitable for causing said support to move with respect to said pivot according to at least two independent directions;
deviating means said at least two forces so that they are applied to said support according to two predetermined positions.

2. Robotic wrist according to claim 1, wherein said support is capable instantaneously to rotate with respect to said fixed pivot so that said support has at least two degrees of freedom with respect to said fixed pivot.

3. Robotic wrist according to claim 1, wherein said at least two forces are applied to said support through means selected from the group:
at least one pulling element, in particular a tendon.
at least one stiff stick in order to act as pulling element and as pushing element.

4. Robotic wrist according to claim 2, wherein, said support can rotate with respect to said fixed pivot according to three degrees of freedom, three pulling elements being provided for applying three respective forces.

5. Robotic wrist according to claim 5, in which said three degrees of freedom of the support are obtained in a redundant way, with four pulling elements for applying four respective forces.

6. Robotic wrist according to claim 5, wherein said deviating means said or each force comprise:
a base, from which said pivot extends, said base being integral to said support, and
a connecting arm between said base and said or each pulling element, the connecting arm being suitable for arranging said pulling element according to a predetermined inclination with respect to said support.

7. Robotic wrist according to claim 5, wherein said or each connecting arm has a first end connected to said base in order to provide a resilient hinge and a second free end connected to a point of said pulling element, whereby when the pulling element moves for actuating the support, the free end of the arm rotates with respect to the first end constraining said point on a circular trajectory.

8. Robotic wrist according to claim 6, where the first end of said or each connecting arm is hinged to said base by a means selected from the group: a flexible lamina; a hinge and a resilient element.

9. Robotic wrist according to claim 1, where the rotating connection between said support and said fixed pivot is effected by a ball joint.

10. Robotic wrist according to claim 9, wherein said pivot has a spherical housing in which a spherical portion integral to said support is housed with freedom of movement.

11. Robotic wrist according to claim 9, wherein said support comprises at least one means of interposition between said distal element and said pivot a portion of which can be deformed in a controlled way with a predetermined combination of forces in order to bring the fixed pivot to contact means for opening/closing said instrument thus causing the opening/closing of said instrument.

12. Robotic wrist according to claim 1, wherein said means for opening/closing said instrument comprise an articulated mechanism having flexible elements.

13. Robotic wrist according to claim 1, comprising a mechanism equivalent to two spheres, or portions of sphere, rolling on each other, wherein said fixed pivot is located at the centre of the first ball and said deviating means provide an arm rotatable about said pivot and connected to the centre of the second sphere, as well as provide said rolling contact between said spheres.

14. Robotic wrist according to claim 13, wherein said mechanism equivalent to two spheres, or portions of sphere, rolling on each other is obtained composing together first and second deviating means comprising each a first and a second kinematical chain comprising each a plurality of stiff elements connected by means of pivot joints and a couple of gears that operate in combination with said first and second kinematical chain.

15. Robotic wrist according to claim 14, wherein said mechanism equivalent to two spheres is obtained replacing said two couples of gears with a tern of stiff elements interconnected by means of pivot joints.

16. Device for teleoperation by means of manipulators “slave” remotely actuated by an operator characterised in that it comprises a robotic wrist according to the previous claims operatively connected by said means for applying said at least two forces, located in said support element having elongated hollow shape, to at least one means for generating said forces.

17. Device according to claim 16, wherein said means for generating said at least one force comprises a motor operatively connected to each connecting arm by said means for applying said force, whereby said robotic wrist is actuated by selectively operating at least one connecting arm or a combination of simultaneous movements of at least two of said connecting arms that cause it to rotation with respect to a determined plane.

18. Device according to claim 16, wherein each transmission means of the force is operatively connected to the respective motor by a pulley connected to the axis of the motor same.

19. Device according to claim 18, wherein each pulley is mounted on a bearing and is associated to a resilient means to it co-axial suitable for allowing the pre-tensioning of said means for applying said force.

20. Device according to claim 18, wherein said motors are associated to sensors of position suitable for determining the position of said robotic wrist and/or of said connecting arms.

21. Device according to claim 15, wherein said motors are operatively connected to said pulleys by a releasable connection.

22. Device according to claim 21, wherein said releasable connection between said motors and said pulleys is effected by means of clutches.

23. Robotic wrist according to claim 1, characterised in that it is used as distal element for mininvasive surgical operations with feedback force.
